(19) United States
(12) Patent Application Publication
(10) Pub. No.: US 2012/0114616 A1
(43) Pub. Date: May 10, 2012

(54) OLFACTORY STEM CELLS AND USES THEREOF

(75) Inventor: Chaker N. Adra, Boston, MA (US)

(73) Assignee: King Faisal Specialist Hospital & Research Centre, Riyadh (SA)

(21) Appl. No.: 13/157,131

(22) Filed: Jun. 9, 2011

Related U.S. Application Data
(63) Continuation of application No. PCT/US2009/006475, filed on Dec. 9, 2009.
(60) Provisional application No. 61/201,427, filed on Dec. 9, 2008.

(51) Int. Cl. A61K 35/12; A61P 19/04; A61P 19/08; C12N 5/071; A61P 25/28

(52) U.S. Cl. 424/93.7; 435/325; 435/366; 435/377

(57) ABSTRACT

Some aspects of this invention are based, at least in part, on the discoveries (i) pluripotent stem cell populations can be obtained from olfactory mucosa, (ii) that various regions of the olfactory mucosa contain pluripotent stem cells, (iii) that cells from these olfactory mucosa derived stem cell populations can be maintained in cultures containing EGF and/or bFGF, (iv) that cells from these olfactory mucosa derived stem cell populations are able to form neurospheres, and/or (v) that cells from these olfactory mucosa derived stem cell populations can differentiate into various lineages, including mesenchymal and neuronal lineages.

### RELATED APPLICATIONS

This application is a continuation of PCT/US2009/006475 filed on Dec. 9, 2009, and claims the benefit of U.S. provisional patent application Ser. No. 61/201,427, filed Dec. 9, 2008 and entitled “Olfactory Stem Cells and Uses Thereof,” the entire disclosures of both of which are incorporated herein by reference.

### FIELD OF THE INVENTION

The invention relates to pluripotent stem cells and uses thereof, including therapeutic applications.

### BACKGROUND OF THE INVENTION

Pluripotent stem cells can be differentiated into various cell lineages and therefore are useful for the treatment of various degenerative or inherited diseases. An accessible source of pluripotent or stem cells would be highly desirable.

### SUMMARY OF THE INVENTION

Some aspects of this invention are based, at least in part, on the discoveries (i) that pluripotent stem cell populations can be obtained from olfactory mucosa, (ii) that various regions of the olfactory mucosa contain pluripotent stem cells, (iii) that cells from these olfactory mucosa derived stem cell populations can be maintained in cultures containing EGF and/or bFGF, (iv) that cells from these olfactory mucosa derived stem cell populations are able to form neurospheres, and/or (v) that cells from these olfactory mucosa derived stem cell populations can differentiate into various lineages, including mesenchymal and neuronal lineages.

Accordingly, some aspects of this invention feature methods of obtaining one or more pluripotent stem cell populations from the olfactory mucosa. Some cells in pluripotent stem cell populations express known stem cell markers, such as tubulin or nestin. According to some aspects of this invention, the expression of stem cell markers, set forth in more detail in the detailed description, can be used to enrich for stem cells in heterogeneous cell populations and/or to derive cell lines, in which a significant percentage of cells expresses one or more stem cell marker. Stem cell lines are generally characterized by their ability to grow in culture without changing their potency, or their differentiation or developmental potential. When given the right molecular cues, at least some of the cells contained in stem cell lines or pluripotent stem cell populations can differentiate into various cell types. In some aspects of this invention, stem cells from the olfactory mucosa differentiate into cell types of the neural and/or mesenchymal lineage.

According to some aspects of the invention, one or more pluripotent stem cell populations are obtained from different regions of the olfactory mucosa, for example the septum or the superior turbinate. The olfactory mucosa is an organ made up of the olfactory epithelium and the lamina propria, or mucus secreting glands, behind the epithelium. The mucus protects the olfactory epithelium and allows odors to dissolve so that they can be detected by olfactory receptor neurons. In mammals, the olfactory mucosa is located on the roof of the nasal cavity above and behind the nostrils.

Some aspects relate to the discovery that pluripotent stem cell populations from the olfactory mucosa can be maintained in vitro. In some embodiments of this invention, the cells are cultured in media containing EGF or bFGF or both. In some embodiments, the cells are cultured in media that does not contain EGF or bFGF. Some aspects feature the formation of neurospheres from cells of pluripotent stem cell populations derived from the olfactory mucosa. Neurospheres are indicative of neuronal stem cells and are a cornerstone during the differentiation of neuronal cell lineages in vitro.

Some aspects of the invention relate to the identification, enrichment, and/or isolation of pluripotent stem cell preparations (e.g., enriched stem cell populations, individual stem cells, stem cell lines, etc.) from the olfactory mucosa of a subject (e.g., of an adult subject, for example an adult human subject). In some embodiments, stem cell preparations are isolated from the lamina propria of the olfactory mucosa of a subject. In some embodiments, stem cell preparations are isolated from the epithelium of the olfactory mucosa (e.g., from a sample of the epithelium from the middle turbinate, superior turbinate, and/or septum of the subject). Some aspects of the invention relate to animal serum free techniques for the culture of mesenchymal and/or neural stem cells. Some aspects of the invention relate to the differentiation of cells of the mesenchymal and/or neuronal lineages from pluripotent stem cell preparations obtained (e.g., enriched or isolated) from the olfactory mucosa. Accordingly, aspects of the invention relate to stem cell preparations (e.g., of about 10, 100, 10³, 10⁴, 10⁵, 10⁶, 10⁷, 10⁸, 10⁹, 10¹⁰, or more cells). In some embodiments, stem cell preparations are enriched (e.g., at least 5%, at least 10%, at least 25%, at least 50%, at least 75%, at least 80%, at least 90%, at least 2 fold, at least 5 fold, at least 10 fold, or more enriched) for one or more stem cells relative to populations of cells from the olfactory epithelium prior to stem cell enrichment and/or in vitro expansion (e.g., prior to cell culture).

According to aspects of the invention, olfactory neural stem cells are useful for differentiation into one or more neural phenotypes (e.g., in vitro or in vivo). In some embodiments, olfactory neural stem cells are useful for transplantation into a subject in need thereof to differentiate into one or more appropriate neural phenotypes after transplantation (e.g., to treat a neurological injury or disorder, for example a neurodegenerative disease or disorder, as described herein). It should be appreciated that in some embodiments, neural and/or mesenchymal stem cell preparations of the invention may be differentiated in vitro prior to transplantation into a subject. However, in some embodiments, one or more stem cell preparations may be transplanted prior to differentiation. It should be appreciated that aspects of the invention may involve injecting an amount of a concentrated population of stem cells that is sufficient to treat a disease or condition or restore or improve one or more physiological functions as described herein.

### BRIEF DESCRIPTION OF THE DRAWINGS

FIG. 1: mesenchymal stem cell markers expressed in cells from adult olfactory mucosa.

FIG. 2: neural stem cell markers expressed in cells from adult olfactory mucosa.

FIG. 3: neurospheres from cells obtained from adult olfactory mucosa.

FIG. 4: Dissociated Olfactory Mucosa cells stained positive for the mesenchymal stem cell markers CD90, CD29, CD166 and CD105. Olfactory cells also stained positive for the neural progenitor marker—nestin, the glial marker—GFAP and the neuronal markers B-III-tubulin and neurofilament.

FIG. 5: Dissociated Bone Marrow cells stained positive for the mesenchymal stem cell markers CD90, CD29, CD166 and CD105. Bone Marrow cells also stained positive for the neuronal markers B-III-tubulin and neurofilament.

FIG. 6: Comparison of the expression of mesenchymal stem cell markers by olfactory mucosa, bone marrow and breast tissue cultures using FACS.

FIG. 7: The expression of mesenchymal stem cell markers (CD105, CD90, CD29 and CD166) in tissue sections of olfactory mucosa also stained with the neural marker neurofilament.

FIG. 8: Bone marrow mesenchymal stem cells differentiate into adipocytes, indicated by the accumulation of lipid, stained with oil-red-O, and the expression of Glut-4.

### DETAILED DESCRIPTION OF THE INVENTION

Some aspects of this invention relate to stem cell populations isolated from the olfactory mucosa. Some cells contained in these cell populations are pluripotent as they can differentiate into various cell types of one or more lineages, for example the mesenchymal lineage and the neural lineage. The mesenchymal lineage comprises fat, cartilage, bone, tendon, ligament, muscle, and skin cells. The neural lineage comprises neurons and glial cells, for example astrocytes. According to some aspects of this invention, the term “pluripotent” qualifies a cell or a cell population as being able to differentiate into more than one cell type. For example a neural stem cell is able to give rise to cell types of both the neuronal and glial lineages, and is accordingly referred to as pluripotent herein. According to some aspects of this invention, some pluripotent stem cell populations obtained from adult olfactory mucosa can be maintained and propagated in culture without loosing their differentiation capacity. According to some aspects of this invention, stem cells or stem cell populations can be frozen to preserve them without substantial loss or change of differentiation potential.

The pluripotent stem cell populations from adult olfactory mucosa according to some aspects of this invention are characterized by the expression of one or more marker genes indicative of pluripotent cells including, but not limited to, the mesenchymal stem cell markers CD105 (isoform 1 precursor: NP_{—}001108225, isoform 2 precursor: NP_{—}000109), CD166 (NP_{—}001618), CD90 (NP_{—}006279), or CD29 (isoform 1A: NP_{—}002202 and NP_{—}596867, isoform 1B: NP_{—}389647, isoform 1Cl: NP_{—}391987, isoform 1C2: NP_{—}391989, isoform 1D: NP_{—}391988) or any combination of these, or the neuronal stem cell markers Tubulin (NP_{—}006077), Neurofilament (light peptide: NP_{—}006149, medium peptide, isoform 1: NP_{—}005373, medium peptide, isoform 2: NP_{—}001099011, heavy peptide: NP_{—}066554) GFAP (isoform 1: NP_{—}002046, isoform 2: NP_{—}001124491), or Nestin (NP_{—}006608) or any combination of two or more of these. The numbers in parentheses are the accession numbers (RefSeq IDs) assigned to these proteins by the National Center for Biotechnology Information (NCBI, http://ncbi.nlm nih.gov), current as of Dec. 8, 2008.

The pluripotent stem cell populations from adult olfactory mucosa according to some aspects of this invention are characterized by containing single cells expressing one or more of the above mentioned marker genes.

Expression of stem cell marker genes can be detected by standard methods well known to those of ordinary skill in the art. Examples of suitable methods include, but are not limited to, immunoassays for the detection of proteins expressed from stem cell marker genes, and nucleic acid detection assays for the detection of RNA transcribed from marker genes. Examples of immunoassays include, but are not limited to, immunohistochemistry, fluorescence activated cell sorting (FACS), magnetic cell sorting (MACS), and western blotting assays. Examples of nucleic acid detection assays include, but are not limited to, polymerase chain reaction (PCR), Reverse transcription polymerase chain reaction (RT-PCR), and northern blot.

The mesenchymal stem cell marker CD105, or endoglin, is a protein expressed in various stem and progenitor cells, for example in osteoprogenitor cells, that can be detected by immunoassay using one of various commercially available antibodies, for example any of those obtainable under the catalog numbers AF1097, MAB10971, MAB 1097, or MAB 10972 from R&D Systems (R&D), Minneapolis, Minn., USA. CD105 is well known to those in the art, see e.g., Ref. 1.

The stem cell marker CD166, or alcam (activated leukocyte cell adhesion molecule), is a protein expressed in mesenchymal stem cells, which can be detected using one of various commercially available antibodies, for example any of those obtainable under the catalog numbers AF656 or MAB656 from R&D. CD166 is well known to those in the art, see e.g., Ref. 2.

The stem cell marker CD90, or thy-1 (thymocyte activated antigen 1), is a 25-37 kDa heavily N-glycosylated, glycophosphatidylinositol (GPI) anchored conserved cell surface protein with a single V-like immunoglobulin domain, originally discovered as a thymocyte antigen. Thy-1 can be used as a marker for a variety of stem cells and for the axonal processes of mature neurons. Thy-1 can be detected using one of various commercially available antibodies, for example one obtainable under the catalog number MAB2067 from R&D. CD90 is well known to those in the art, see e.g., Ref. 3.

The stem cell marker CD29, or integrin 1 beta, or B 1-integrin, is a mesenchymal stem cell marker. CD 29 can be detected using one of various commercially available antibodies, for example any of those obtainable under the catalog numbers MAB 1778, AF1778, or MAB17783 from R&D. CD29 is well known to those in the art, see e.g., Ref. 4.

The stem cell marker Stro-1 antigen is expressed in mesenchymal stem and precursor cells able to give rise to various cell types, comprising adipocytes, osteocytes, smooth myocytes, fibroblasts, and chondrocytes. Stro-1 can be detected using one of various commercially available antibodies, for example any of those obtainable under the catalog numbers MAB1038, or FAB1038F from R&D. Stro-1 is well known to those in the art, see e.g., Ref. 5.

The stem cell marker Nestin is an intermediate filament structural protein expressed in neural stem cells. Nestin can be detected using one of various commercially available antibodies, for example any of those obtainable under the catalog numbers MAB 1259 or IC1259F from R&D. Nestin is well known to those in the art, see e.g., Ref. 6, 7, 8, 9, 10, and 11.

The neuronal cell markers neural tubulin, or beta tubulin III, or tuj-1, and neurofilament, are structural proteins important for neuron function and are indicative of more differentiated neuronal cells. Beta tubulin can be detected using one of various commercially available antibodies, for example any of those obtainable under the catalog numbers ab7792, ab52623, or ab15568 from Abcam Inc. (Abcam), Cambridge, Mass., USA. Neurofilament can be detected using one of various commercially available antibodies, for example any of those obtainable under the catalog numbers ab59427, ab7795, ab9034, or ab9035 from Abcam. Beta tubulin and neurofilament are well known to those in the art, see e.g., Ref. 11.

The glial cell marker glial fibrillary acidic protein (GFAP) is an intermediate filament protein specifically produced by glial cells, for example by astrocytes. GFAP can be detected using one of various commercially available antibodies, for example any of those obtainable under the catalog numbers MAB2594 or AF2594 from R&D. GFAP is well known to those in the art, see e.g., Ref. 11 and 12.

In some embodiments of this invention, only one of stem cell markers described herein may be expressed in a stem cell preparation (e.g., an enriched stem cell population, a stem cell line, single stem cells, etc.) of the invention. However, in certain embodiments, combinations of two or more of the stem cell markers (e.g., 2, 3, 4, 5, 6, 7, 8, 9, or 10) are expressed in a preparation of stem cells derived from the olfactory mucosa (e.g., an enriched stem cell population, a stem cell line, single stem cells, etc.). Examples of combinations include, but are not limited to, (i) CD105, CD166, and CD90, (ii) CD105, CD 166, CD90, and CD29, (iii) tubulin, neurofilament, and GFAP, (iv) tubulin, neurofilament, GFAP and nestin, (v) GFAP and nestin, (vi) tubulin, GFAP and nestin, (vii) a combination of (i) and (iii), (viii) a combination of (ii) and (iii), (ix) a combination of (i) and (iv), (x) a combination of (ii) and (iv), and (xi) a combination of (ii) and (v).

Pluripotent stem cell populations according to some aspects of this invention can be obtained from samples extracted from at least three different regions of the olfactory mucosa, the superior turbinate, the middle turbinate and the septum.

In some embodiments of the invention, a sample from the olfactory mucosa is explanted. In some embodiments, a sample is dissociated, for example by means of an enzymatic dissociation. The term “explanted, as used herein, is intended to include, but not to be limited to, placing a biopsy sample directly into a culture dish. Cells contained in said biopsy sample are allowed to attach to the surface of said culture dish. However, they are not required to attach. The term “enzymatic dissociation”, as used herein, is intended to include, but not to be limited to, dissociation of a biopsy sample, in full or in part, by contacting said biopsy sample with an enzyme or a composition of enzymes digesting extracellular matrix components and/or cell-cell contact proteins or, in some other way, weakening tissue cohesion and/or integrity, resulting in the separation of single cells or cell aggregates from said biopsy sample. Examples of suitable enzymes are collagenase and trypsin. However, as is well known to those of skill in the art, other enzymes may be used and the invention is not limited in this respect.

Some pluripotent stem cell populations derived from olfactory mucosa according to this invention can be cultured in vitro. In some embodiments of this invention the cells are cultured under conditions well known to those of ordinary skill in the art allowing at least some cells to maintain a pluripotent stem cell state and sustained self-renewal.

In some embodiments, cells are cultured in the presence of growth factors. In some embodiments, cells are cultured in the presence of epidermal growth factor (EGF). In some embodiments, cells are cultured in the presence of basic fibroblast growth factor (bFGF). In some embodiments, cells are cultured in the presence of EGF and bFGF. However, cells may be cultured in the presence of one or more other growth factors or in the absence of these growth factors, as the invention is not limited in this respect.

According to some aspects of the invention, at least some cells of a pluripotent stem cell population obtained from the olfactory mucosa can differentiate into cells of one or more of various lineages, for example the mesenchymal or neuronal lineages or both. In some embodiments of this invention, cells are cultured under conditions inducing at least some cells to differentiate into cells of either the mesenchymal lineage or the neural lineage or both.

In some embodiments of this invention, cells are cultured in the presence of EGF and/or bFGF under conditions allowing at least some cells to form neurospheres. Neurospheres can be cultivated by cell culture techniques well known to those of skill in the art.

In an exemplary method for culturing olfactory stem- and progenitor cells, biopsies are immediately placed on ice in DMEM/HAM F12 supplemented with 10% fetal calf serum, penicillin, and streptomycin and then incubated for 15-45 min at 37° C. in a 1-5 units/ml Dispase II solution (Boehringer). Laminae propriae are carefully separated from the epithelium under a dissection microscope. Sheets of olfactory epithelium can be mechanically dissociated while lamina propriae are cut into pieces. In some embodiments, this dissociation is followed by incubation in a collagenase solution, e.g. 25 mg/ml, for 5-20 min at 37° C. After mechanical trituration, the enzymatic activity can be stopped, for example using a 0.5 mM ethylenediaminetetraacetic acid solution. In some embodiments, cell pellets of both tissues are resuspended in DMEM/HAM F12 culture medium containing 10% fetal calf serum and plated into culture vessels pretreated with poly-L-lysine (e.g., 1 μg/cm 2). 12-24 hours after initial plating, floating cells and undigested pieces of epithelium and lamina propria can be transferred to coated wells. Spheres of cells can subsequently be harvested collectively by aspiration of the culture medium and subsequent centrifugation or individually using a small pipette.

Cell spheres can be plated on glass or plastic dishes with a coating allowing for cell adhesion, e.g., a coating of collagen IV (5 μg/cm 2), fibronectin (10 μg/cm 2), laminin (3.5 μg/cm 2), poly-L-lysine (2 μg/cm 2), or poly-ornithine (10 μg/cm 2).

In some embodiments, the media is supplemented with a growth factor, for example with bFGF and/or EGF.

In some embodiments, olfactory stem cells are cultured under conditions well known to those in the art suitable for neural stem or progenitor cell culture. In some embodiments, a serum-free medium (SFM) is sed for olfactory stem cell culture. In some embodiments, the basic SFM is DMEM/F12, at a ratio of 1:1 (volume) supplemented with L glutamine and HEPES. Additional components added to the SFM include, in some embodiments, putrescine (1,4-Diaminobutane dihydro-chloride) 0.0096 g/l progesterone 0.0000629 g/l, B-27 Supplement, Insulin-Transferrin-Sodium Selenite Supplement (ITSS), Heparin, Trypsin inhibitor, and growth factors.

In some embodiments, the growth factors included in the medium are fibroblast growth factor-basic (bFGF), and epidermal growth factor (EGF),

In some embodiments, cells and/or cell spheres isolated from olfactory tissue (and/or umbilical cord and/or bone marrow) can be grown under conditions suitable for promoting and/or propagating neurospheres (e.g., as described in Reynolds B A and Weiss S (1992). “Generation of neurons and astrocytes from isolated cells of the adult mammalian central nervous system”. Science 255: 1707-1710). It should be appreciated that any suitable growth condition for promoting and/or propagating neurospheres and/or neural stem cells may be used.

In some embodiments, cells and/or cell spheres isolated from olfactory tissue (and/or umbilical cord and/or bone marrow) can be grown under conditions suitable for promoting and/or propagating mesenchymal cells. In some embodiments, a CFU-f approach may be used, where isolated cells are plated directly into cell culture plates or flasks. Mesenchymal stem cells are adherent to tissue culture plastic within 24 to 48 hours. In some embodiments, a direct-plating technique analogous to methods used for bone marrow may be used (e.g., as described in Concise review: mesenchymal stem/multipotent stromal cells: the state of transdifferentiation and modes of tissue repair—current views. Stem Cells. 2007 Nov;25(11):2896-902, Epub 2007 Sep 27; or in Wan C, He Q, McCaigue M, Marsh D, Li G (2006), Nonadherent cell population of human marrow culture is a complementary source of mesenchymal stem cells (MSCs), Journal of Orthopaedic Research 24 (1): 21-8). Other methods (e.g., flow cytometry-based methods may be used to sort cells for specific surface markers, e.g., STRO-1, or any one or more mesenchymal markers described herein).

It should be appreciated that any suitable growth condition for promoting and/or propagating mesenchymal stem cells may be used.

According to certain embodiments, neuronal and/or mesenchymal stem cells may be differentiated (e.g., in vitro) using any suitable technique known in the art. For example, mesenchymal stem cells may be differentiated as described in U.S. Pat. No. 5,197,985; US patent publication 20090232777, or by Dalby et al., Nat. Mater., 2007 Dec, 6(12): 997-1003, The control or human mesenchymal cell differentiation using nanoscale symmetry and disorder. For example, neural stem cells may be differentiated as described in U.S. Pat. No. 6,033,906; U.S. Pat. No. 7,211,434; or US patent publication 20080206865. However, it should be appreciated that any suitable condition or technique may be used for differentiating mesenchymal and/or neural stem cells as aspects of the invention are not limited in this respect.

Cells and cell spheres (differentiated and/or undifferentiated) can be cryopreserved using methods well known to those in the art. For example, spheres can be frozen in 90% serum/10% dimethyl sulfoxide or any other cryopreservation medium known to those in the an to be suitable for freezing mesenchymal or neural stem cells. However, other techniques may be used.

Cells and cell spheres can be preserved for later use or immediately plated on glass or plastic dishes, e.g., without or with a coating of collagen IV, fibronectin, laminin, poly-L-lysine, and/or poly-ornithine.

In some aspects of this invention, cells from neurospheres are cultured under conditions well known to those of ordinary skill in the art inducing at least some of these cells to differentiate into cell types of the neuronal and/or glial lineages.

In some embodiments of the invention, cell populations obtained from olfactory mucosa are enriched for pluripotent stem cells by sorting primary cell populations for or against cells expressing any one or a combination of the marker genes CD105, CD166, CD90, CD29, Stro1, Tubulin, Neurofilament, GFAP, or Nestin or any combination of these. This sorting can be performed by standard methods well known to those of skill in the art, for example fluorescence activated cell sorting (FACS) or magnetic cell sorting (MACS) or any other suitable method, as the invention is not limited in this respect.

Some embodiments of the invention feature preparations of cultured pluripotent stem cell populations obtained from the olfactory mucosa. At least some of the cell contained in these are able to self-renew in vitro and their frequency in a cell culture is substantially stable over several cell passages. For example, in some embodiments, the percentage of stem cells is less than 1%, in other embodiments, the percentage is about 1%, about 2%, about 5%, about 5-10%, about 10%, about 10-20%, about 20-30%, about 25-50%, about 25%, about 30%, about 40%, about 50%, about 50-75%, about 60%, about 70%, about 75%, about 75-100%, about 80%, about 90%, about 95%, about 99%, or about 100%.

Some embodiments of this invention feature one or more uncultured pluripotent stem cell populations from the olfactory mucosa.

In some aspects, preparations of one or more pluripotent stem cell populations, e.g., one or more stem cell lines, are obtained from a human subject. In some aspects, one or more pluripotent stem cell preparations of the invention are administered to a human subject. In some embodiments, the human subject (e.g., from which stem cells are obtained, and/or to which stem cells are administered) is indicated to have one or more diseases or conditions. In some embodiments, at least one of this one or more diseases or conditions is a chronic disease or condition. In some embodiments, at least one of this one or more diseases or conditions is a degenerative disease or condition. Examples of degenerative diseases include, but are not limited to, Atherosclerosis, Rheumatoid Arthritis, Osteoporosis, Osteoarthritis, neurodegenerative diseases, and Diabetes. In some embodiments, at least one of this one or more diseases or conditions is a neurodegenerative disease or condition. Examples of neurodegenerative diseases or conditions include, but are not limited to, Alzheimer's disease, Creutzfeldt-Jakob disease, Huntington's disease, Parkinson's disease, ALS, Multiple Sclerosis, and Spinal muscular atrophy. In some embodiments, the subject is an orthopedic patient.

According to some aspects of this invention, cells are differentiated into cell types of at least one of various lineages, for example the mesenchymal lineage or the neuronal lineage, or both.

In some embodiments of the invention, at least some cells of a pluripotent stem cell population obtained from the olfactory mucosa, or cells differentiated from a pluripotent stem cell population are administered to a subject. In some embodiments, the donor and the recipient of cells or cell populations is the same subject. In some embodiments, the genotype of cells or cell populations is determined before they are administered to a subject. In some embodiments, the determination of the genotype comprises a determination of the presence of a viral infection in these cells. In some embodiments, the determination of the genotype comprises a determination of the human leukocyte antigen (HLA). In some embodiments, cells to be administered have been found to have a genotype, for example a HLA genotype compatible with the genotype of the recipient of the cells. In some embodiments, a genetic mutation prevalent in said subject's cells is repaired in said cells before administering them to said subject. For example, a mutation causing Huntington's disease might be repaired in affected cells by targeting the mutated Huntingtin allele with a correct copy of the gene, selecting cells carrying the corrected version and administering these cells, or their differentiated progeny to a subject indicated to have Huntington's disease. This strategy is applicable to various genetic diseases as the invention is not limited in this respect.

In some embodiments, one or more pluripotent stem cell populations, e.g., one or more stem cell lines, are obtained from the olfactory mucosa of a subject. In certain embodiments, this one or more population and/or cell line comprises cells expressing one or more mesenchymal stem cell marker genes and/or one or more neural stem cell marker genes. In some embodiments, these stem cell marker genes comprise one or more of the genes CD 105, CD 166, CD90, CD29, Tubulin, Neurofilament, GFAP, Nestin, or any combination of them. In some embodiments, this one or more cell population and/or one or more cell line comprises cells negative for Stro 1 expression (e.g., in an immunoassay). However, in some embodiments, stem cells may be positive for Stro 1 expression (e.g., in an immunoassay).

According to some aspects of the invention, the pluripotent stem cell population obtained from the olfactory mucosa comprises cells that expand rapidly. According to some aspects of the invention, at least some cells of the pluripotent stem cell population are able to form neurospheres. According to some aspects of this invention, cells of said pluripotent stem cell population are able to differentiate into cells of the mesenchymal and/or neuronal lineage. The pluripotent stem cell population according to some aspects of the invention comprises cells that are able to differentiate into adipocytes, osteocytes, chondrocytes, neuronal cells, or glial cells.

In some embodiments, this invention provides compositions comprising at least one pluripotent stem cell population obtained from the olfactory mucosa of a subject.

In some embodiments, this invention provides methods of administering at least one pluripotent stem cell population, obtained by any of the methods described herein or at least one of any of the pluripotent stem cell populations from the olfactory mucosa described herein or any composition containing pluripotent stem cell populations as described herein, with the intent to ameliorate a disease or condition in said subject.

In some embodiments, one or more pluripotent stem cell populations, e.g., one or more stem cell lines, are derived from the olfactory mucosa of a subject and cells contained within this one or more pluripotent stem cell population, or the differentiated progeny of at least one stem cell, are returned to the subject, thus avoiding the problems associated with immune rejection of foreign transplanted cells or tissues. In some embodiments, these cells are expanded and/or genetically or otherwise manipulated in culture before they are returned to the subject. In some embodiments, this manipulation is genetic manipulation by methods well known to those of skill in the art, for example gene targeting, viral gene transfer, gene or DNA knock-in or knock-out technology or random transgenic insertion. In some embodiments, donor and recipient of cells are the same subject. In some embodiments, donor and recipient are not the same subject.

In some embodiments of this invention a subject receiving cells of the pluripotent stem cell population, or differentiated progeny thereof, has or is suspected to have one or more diseases or conditions, including, but not limited to, spinal cord injury, acute insult to peripheral neurons, neurodegenerative disease, or bone or cartilage disease or dysfunction.

Some embodiments of this invention relate to cell populations comprising adult stem cells. In some embodiments, stem cells of the invention are not human embryonic stem cells.

The terms “cell population” and “cell line”, as used herein according to this invention, refer to populations of isolated cells. The term “cell population” is intended to refer to, but not to be limited to, any distinct group of cells isolated or derived from a biological sample. The term “cell population” is intended to refer to, but not to be limited to, a cell culture, a frozen cell sample, a selected group of cells, wherein examples of selection are by resistance or sensitivity to chemical compounds, including antibiotic agents, by marker gene expression, and various other suitable methods. The term “cell population” is intended to refer to, but not to be limited to, both heterogeneous and homogeneous cell populations. The term “cell line” is intended to refer to, but not to be limited to, homogeneous populations of cells that can be propagated without substantially changing their characteristics, e.g., their morphology, frequency of cell division, marker gene expression profile or differentiation potential. Accordingly, a cell line is an example of a cell population.

The term “pluripotent stem cell population”, as used herein, is intended to refer to any population of stem cells derived, directly or indirectly, from the olfactory mucosa of a subject. The term “pluripotent stem cell populations”, as used herein, is intended to refer to, but not to be limited to, populations derived from different biopsies from different subjects, populations from different biopsies from the same subject, or populations from the same biopsy from the same subject. In some embodiments, the term “pluripotent stem cell populations” is further intended to refer to, but not to be limited to, two or more distinct cell populations generated from a single biopsy sample or cell culture derived from a biopsy sample by splitting a biopsy sample or cell culture derived from a biopsy sample into more than one culture dish, freezing a biopsy sample or cell culture derived from a biopsy sample into more than one freezing vessel, or in any way separating subpopulations of cells from the original biopsy sample or cell culture derived from a biopsy sample.

The term “treatment” or “treating” is intended to include one or more of prophylaxis, amelioration, prevention or cure of a condition (e.g., Alzheimer's disease). Treatment after a condition (e.g., Alzheimer's disease) has been diagnosed or clinically manifested aims to reduce, ameliorate or altogether eliminate the condition, and/or its associated symptoms, or prevent it from becoming worse. Treatment of subjects before a condition (e.g., Alzheimer's disease) has been diagnosed or clinically manifested (e.g., prophylactic treatment) aims to reduce the risk of developing the condition and/or lessen its severity if the condition does develop. As used herein, the term “prevent” refers to the prophylactic treatment of a subject who is at risk of developing a condition (e.g., Alzheimer's disease) resulting in a decrease in the probability that the subject will develop the disorder, and to the inhibition of further development of an already established disorder.

As used herein, a treatment may be prophylactic and/or therapeutic. In some embodiments, a treatment may include preventing disease development or progression. In certain embodiments, a treatment may include inhibiting and or reducing the rate of disease development or progression. It should be appreciated that the terms preventing and/or inhibiting may be used to refer to a partial prevention and/or inhibition (e.g., a percentage reduction, for example about 5%, about 10%, about 20%, about 30%, about 40%, about 50%, about 60%, about 70%, about 80%, about 90%, or higher or lower or intermediate percentages of reduction). However, in some embodiments, a prevention or inhibition may be complete (e.g., a 100% reduction or about a 100% reduction based on an assay or an expected progression).

While it is possible for one or more stem cell populations, or derivatives thereof, of the present invention to be administered alone, in general embodiments one or more cell populations, or derivatives thereof, may be administered as a pharmaceutical formulation (composition) as described herein. Evident to one of ordinary skill in the art, compositions may comprise a suitable buffer or solvent that is physiologically compatible.

One or more stem cell populations, or derivatives thereof, according to some aspects of the invention may be formulated for administration in any convenient way for use in human or veterinary medicine, by analogy with other pharmaceuticals.

Some aspects of the invention relate to a method of making a medicament for use in treating a subject at risk for developing or having or suspected of having a disease or condition, e.g., a subject at risk for developing or having or suspected of having Huntington's disease or other disease described herein. Medicaments can be used for prophylactic treatment of a subject at risk for developing or suspected of having a disease, e.g., Huntington's disease or other disease described herein (e.g., for treatment of a subject prior to, during, and/or after the subject displays any symptoms). Accordingly, one or more stem cell populations, e.g., one or more stem cell lines, or derivatives thereof, or compositions as described herein may be used for the preparation of a medicament for use in any of the methods of treatment described herein. In some embodiments, the invention provides for the use of one or more stem cell populations, e.g., one or more stem cell lines, or derivatives thereof, for the manufacture of a medicament or pharmaceutical for treating a subject (e.g., a human) having one or more symptoms of, or being at risk for developing a disease or condition, e.g., Huntington's disease or other disease described herein. Accordingly, some aspects of the invention relate to the use of one or more stem cell populations, or derivatives thereof, as described herein for the preparation of a medicament for treating or preventing a disease or condition, as described herein, in a subject.

It also should be appreciated that in some embodiments, aspects of the invention relate to isolating mesenchymal and/or neural stem cells from bone marrow, and/or umbilical cord in addition to or instead of from olfactory mucosa. It should be appreciated that one or more methods or techniques described herein in the context of cells isolated from olfactory mucosa may be used in connection with cells (e.g., neuronal progenitor cells) isolated from bone marrow and/or umbilical cord.

In certain embodiments, aspects of the invention relate to using either VCAM and/or ICAM alone or in combination with one or more additional markers described herein as discriminating markers between cells isolated from bone marrow and olfactory mucosa.

In some embodiments, aspects of the invention relate to isolating olfactory ensheathing cells from olfactory mucosa (e.g., using FACS or other isolating method, for example, involving CD166 antibodies to identify and/or purify the ensheathing cells). Isolated ensheathing cells may be propagated. Isolated ensheathing cells (either propagated or non-propagated) may be used in a cell therapy (e.g., by injecting them into a subject at a site of spinal cord injury, or at a site of other injury or tissue disease).

It should be appreciated that cells (e.g., neural and/or mesenchymal) may be isolated from tissue (e.g., olfactory tissue, bone marrow, and/or umbilical cord) using affinity techniques (e.g., based on antibodies) against one or more neural and/or mesenchymal markers (e.g., 1, 2, 3, 4, 5, . . . or all) described herein, including, but not limited to, those exemplified in the examples. In some embodiments, a cell sorting technique (e.g,. a FACS based technique) may be used to enrich, isolate, and/or purify cells of interest (e.g., prior to, as a part of, or after, propagation in culture) based on binding (e.g., of labeled antibodies or other ligands) to one or more markers characteristic of the cells described herein.

Accordingly, in some embodiments, a tissue sample (e.g., olfactory tissue) is obtained, dissociated, and the cells are optionally propagated and/or differentiated in culture. In some embodiments, an isolation (or enrichment or purification) step is used (e.g., prior to, during, or after, propagation and/or differentiation).

It should be appreciated that cells described herein may be used in research and/or therapy. In research, the cells may be tested to determine the extent to which they can form different tissue types that may be therapeutically useful. The cells also may be tested for responses to different conditions or drugs, etc., or any combination thereof.

In therapy, cells may be injected into a subject (e.g., a human subject) at a site where differentiated cells are needed (e.g., due to an injury or a disease, such as a degenerative disease, or other condition).

Recent investigations into the treatment of spinal cord injuries using stem cell therapy have shown promising results. The majority of studies utilize non-neural tissue as a source of stem cells. In particular, mesenchymal stem cells have been utilized in transplantation experiments to treat animal models of neural disorders. Upon transplantation mesenchymal stem cells allow functional improvement by providing immunosuppressive and neurotrophic support. However, mesenchymal stem cells, largely, fail to differentiate when transplanted. According to aspects of the invention, the human olfactory epithelium is an accessible source of stem cells. The lamina propria of the olfactory mucosa contains mesenchymal tissue that may house mesenchymal stem cells. The epithelium of the olfactory mucosa may be used to obtain and isolate neural stem cell populations. As an inherently neural stem cell population, olfactory neural stem cells are expected to be more likely to differentiate into appropriate neural phenotypes upon transplantation than non-neural stem cells. Therefore, the human olfactory mucosa may be a unique source of both neural stem cells and mesenchymal stem cells for therapeutic use according to aspects of the invention. In some embodiments, treatment of spinal cord injuries may be improved by using both neural and mesenchymal stem cells derived from the human adult olfactory mucosa in combination. It also should be appreciated that a combination of neural and mesenchymal stem cells isolated from other sources (e.g., umbilical cord, and/or bone marrow) also may be used (e.g., alone or along with those derived from olfactory tissue. It also should be appreciated that one or more stem cells and/or combinations of stem cells may be used to treat other conditions as described herein.

Cells may be administered, such as by injection, to a patient in any setting in which a disease or condition occurs that can be treated with neuronal or mesenchymal stem cells or a combination thereof. The cells may be extracted in advance and stored in a cryopreserved fashion or they may be extracted at or around the time of defined need. As disclosed herein, the cells may be administered to the patient, such as, for example, by injection, or applied directly to diseased or damaged tissue, such as, for example, by injection, or in proximity of the damaged tissue, such as, for example, by injection, without further processing or following additional procedures to further purify, modify, stimulate, or otherwise change the cells. For example, the cells obtained from a patient may be administered (e.g., injected) to a patient in need thereof without culturing the cells before administering them to the patient. However, in some embodiments, cells may be propagated and/or differentiated prior to administration as described herein.

In accordance with aspects of the invention, regenerative stem cells can be delivered to the patient soon after harvesting the cells from the patient. For example, the cells may be administered immediately after the processing of patient tissue (e.g., olfactory tissue) to obtain a composition of regenerative cells. In some embodiments, a tissue sample is analyzed as described herein (e.g., to detect the presence of one or more markers described herein) and if the desired markers are present, the cell population may be administered to the patient. In another embodiment, the timing for delivery may be relatively longer if the cells to be re-infused to the patient are subjected to additional modification, purification, stimulation, or other manipulation, as discussed herein. Furthermore, the regenerative stem cells may be administered multiple times. For example, the cells may be administered continuously over an extended period of time (e.g., hours), or may be administered in multiple bolus injections extended over a period of time. In certain embodiments, an initial administration of cells will be administered within about 12 hours, such as at 6 hours, and one or more doses of cells will be administered at 12 hour intervals.

The number of cells administered to a patient may be related to, for example, the cell yield after tissue processing. A portion of the total number of cells may be retained for later use or cyropreserved. In addition, the dose delivered will depend on the route of delivery of the cells to the patient. In one embodiment of the invention, the number of regenerative cells to be delivered to the patient is expected to be about 10⁵ cells. However, this number can be adjusted by orders of magnitude (e.g., 1, 2, 3, or more orders of magnitude higher or lower) to achieve the desired therapeutic effect.

Cells may also be applied with additives to enhance, control, or otherwise direct the intended therapeutic effect. For example, in one embodiment, and as described herein, the cells may be further purified by use of antibody-mediated positive and/or negative cell selection to enrich the cell population to increase efficacy, reduce morbidity, or to facilitate ease of the procedure. Similarly, cells may be applied with a biocompatible matrix which facilitates in vivo tissue engineering by supporting and/or directing the fate of the implanted cells. In the same way, cells may be administered following genetic manipulation such that they express gene products that are believed to or are intended to promote the therapeutic response(s) provided by the cells. Examples of manipulations include manipulations to control (increase or decrease) expression of factors promoting growth and/or differentiation, e.g., angiogenesis or vasculogenesis (for example VEGF). Cells may also be subjected to cell culture on a scaffold material prior to being implanted.

In some embodiments, indirect administration of cells to the site of intended benefit is preferred. This may be achieved through a peripheral intravenous injection. Routes of administration known to one of ordinary skill in the art, include but are not limited to, intravenous, intra-arterial, and may or may not include an endo-vascular catheter based mechanism of delivery.

Cells may be injected in a single bolus, through a slow infusion, or through a staggered series of applications separated by several hours or, provided cells are appropriately stored, several days or weeks. Cells may also be applied by use of catheterization such that the cells are delivered directly to the region of affected tissue. As with peripheral venous access, cells may be injected through the catheters in a single bolus or in multiple smaller aliquots. Cells may also be applied directly to the affected tissue at the time of an operation (e.g., during surgery).

In some embodiments, the route of delivery will include intravenous delivery through a standard peripheral intravenous catheter, or a central venous catheter. The flow of cells may be controlled by serial inflation/deflation of distal and proximal balloons located within the patient's vasculature, thereby creating temporary no-flow zones which promote cellular engraftment or cellular therapeutic action. Furthermore, cells could be delivered through the following routes, alone, or in combination with one or more of the approaches identified above: subcutaneous, intramuscular, and sublingual.

In some embodiments, the stem cells that are administered to a patient can act as growth factor delivery vehicles. For example, by engineering the cells to express one or more suitable growth factors, the cells can be administered to a patient, and engineered to release one or more of the growth factors. The release can be provided in a controlled fashion for extended periods of time. For example, the release can be controlled so that the growth factor(s) are released in a pulsed or periodic manner such that there are local elevations in growth factor, and/or local recessions in the amount of growth factor in proximity to an affected area of tissue. Similarly, stem cells of the invention may be engineered to express one or more other proteins or molecules of interest (e.g., of therapeutic benefit).

It should be appreciated that the cells that are administered to a patient not only help restore function to damaged or otherwise unhealthy tissues, but also facilitate remodeling of the damaged tissues. Cell delivery may take place but is not limited to the following locations: clinic, clinical office, dialysis center, emergency department, hospital ward, intensive care unit, operating room, catheterization suites, and radiologic suites. In some embodiments, the effects of cell delivery therapy would be demonstrated by, but not limited to, an improvement of one or more clinical measures of a condition described herein. The effects of cellular therapy can be evident over the course of days to weeks after the procedure. However, beneficial effects may be observed as early as several hours after the procedure, and may persist for several years. Patients are typically monitored prior to and during the deliver of the cells.

Accordingly, some aspects of the invention relate to one or more stem cell populations, or derivatives thereof, of the invention for use as a medicament. Some aspects of the invention relate to one or more stem cell populations, or derivatives thereof, for use in methods of the invention, for example in methods of treating or preventing a degenerative disease.

As used herein, a “subject” can be a human, non-human primate, or other mammal, e.g., cow, horse, pig, sheep, goat, dog, cat or rodent.

These and other aspects of the invention are illustrated by the following non-limiting examples. It should be appreciated that in some aspects one or more embodiments described in the examples may be generally applicable in combination with one or more embodiments described above.

### EXAMPLES

### Example 1

Extraction of Adult Olfactory Mucosa

Adult olfactory biopsies were obtained by routine nasal surgical methods. From eight patients, 24 biopsies were obtained, one each from the middle turbinate, the superior turbinate and the septum from each patient. Biopsies were either directly explanted into dissociated either mechanically or enzymatically, for example by collagenase treatment.

Immunohistochemistry

Cells were cultured using standard tissue culture procedures well known to those of ordinary skill in the art Immunohistochemistry was performed using standard procedures well known to those of ordinary skill in the art.

Neurosphere Formation Assay

For assaying the ability of cells to form neurospheres, cells were cultured in media containing EGF and bFGF, a procedure well known to those of ordinary skill in the art. Neurosphere formation was assayed

Mesenchymal Stem Cells from Adult Olfactory Mucosa.

Cells from 24 adult olfactory mucosa biopsies from the middle turbinate (A), the superior turbinate (B) and the septum (C) were explanted or enzymatically dissociated (Table 1). Rapidly growing cultures of cells were obtained from adult olfactory mucosa biopsies and cells were assayed for morphology and stem cell marker expression after culture.

TABLE 1
Summary of olfactory biopsies

| | Method of | Olfactory | # of frozen |
|---|---|---|---|
| Patient # | Processing | location | samples |
| OLF1 | Explant | A | 37 |
| | | B | 17 |
| | | C | 11 |
| OLF2 | Explant | A | 5 |
| | | B | 12 |
| | | C | 3 |
| OLF3 | Explant | A | 4 |
| | | B | 15 |
| | | C | 3 |
| OLF4 | Explant | A | 14 |
| | | B | 28 |
| | | C | 23 |
| OLF5 | Enzyme | A | 6 |
| | Dissociation | B | 31 |
| | | C | 15 |
| OLF6 | Explant | A | 4 |
| | | B | 3 |
| | | C | 0 |
| OLF7 | Enzyme | A | n/a |
| | Dissociation | B | n/a |
| | | C | n/a |
| OLF8 | Explant | A | n/a |
| | | B | n/a |
| | | C | n/a |

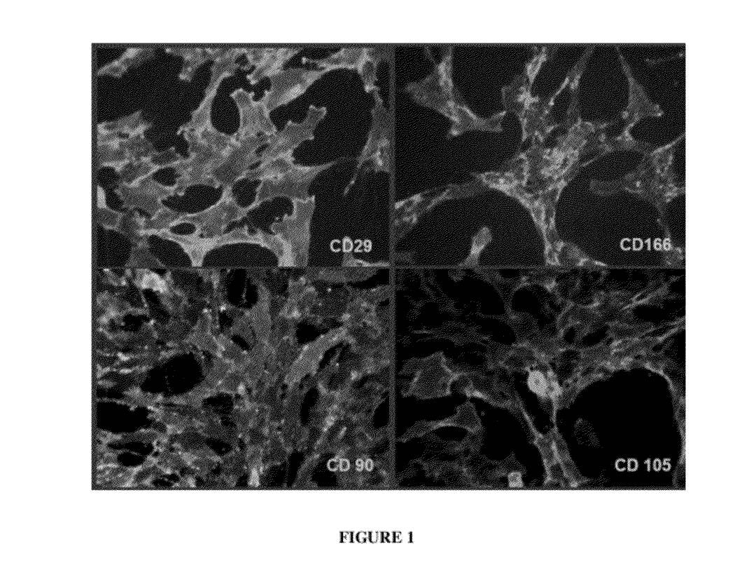

Cell cultures obtained from a superior turbinate biopsy and a middle turbinate biopsy of patient 1, a middle turbinate biopsy of patient 6 and a superior turbinate biopsy of patient 4 were examined (Table 2). Cells were found that morphologically resembled mesenchymal stem cells and expressed the mesenchymal stem cell markers CD29, CD90, CD105 and CD166 (FIG. 1). Cells were found not to express Stro-1.

TABLE 2
Summary of mesenchymal stem cell marker expression
in cell cultures derived from olfactory biopsies

| Marker | OLF1 A | OLF1 B | OLF6 B | OLF4 A |
|---|---|---|---|---|
| CD105 | + | + | + | + |
| CD166 | + | + | + | + |
| CD90 | + | + | + | + |
| CD29m | n/t | n/t | + | + |
| Stro-1 | − | − | − | − |

### Example 2

Neuronal Stem Cells from Adult Olfactory Mucosa

Cells from 24 adult olfactory mucosa biopsies from the middle turbinate, the superior turbinate and the septum were explanted or enzymatically dissociated (Table 1). Rapidly growing cultures of cells were obtained from adult olfactory mucosa biopsies and cells were assayed for morphology and stem cell marker expression after culture.

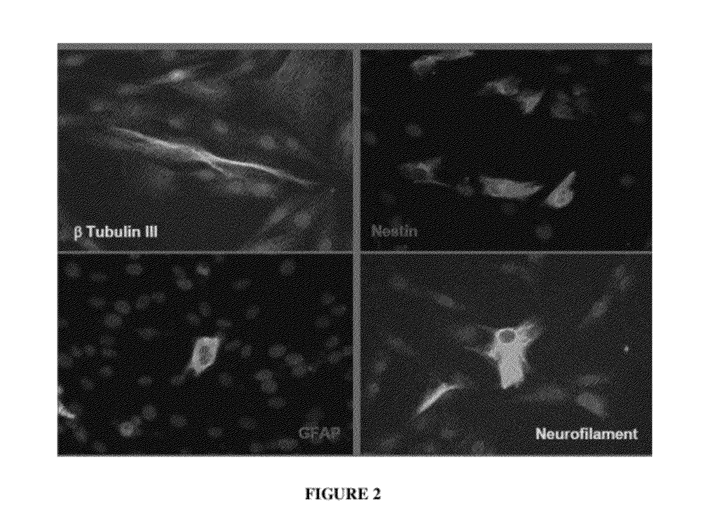

Cell cultures obtained from a superior turbinate biopsy and a middle turbinate biopsy of patient 1, a middle turbinate biopsy of patient 6 and a superior turbinate biopsy of patient 4 were examined (Table 3). Cells were found that morphologically resembled neural stem cells, neural precursor cells, neurons and glia and to express the neural stem cell markers and/or neural markers beta-tubulin III, nestin, GFAP, and neurofilament (FIG. 2).

TABLE 3
Summary of neural stem cell marker expression in
cell cultures derived from olfactory biopsies

| Marker | OLF1 A | OLF1 B | OLF6 B | OLF4 A |
|---|---|---|---|---|
| Tubulin | + | + | + | ? |
| Neurofilament | + | + | + | − |
| GFAP | + | + | + | + |
| Nestin | n/t | n/t | + | + |

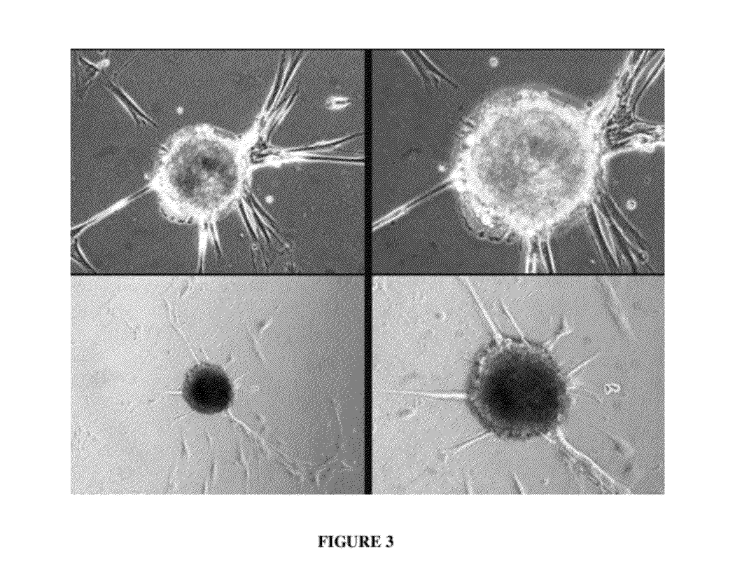

The cells obtained from olfactory mucosa were cultured in media containing EGF and/or bFGF and were able to form neurospheres, indicative of neural stem cells (FIG. 3).

### Example 3

### Marker Expression in Stem Cell Populations from Olfactory Mucosa, Bone Marrow, and Umbilical Cord

Sample Collection

Samples were collected from bone marrow, umbilical cord and olfactory mucosa. Bone marrow and umbilical cord are known to contain mesenchymal stem cells. Therefore these known sources of mesenchymal stem cells can be compared with olfactory mucosa to assess whether this tissue contains mesenchymal stem cells.

3 Bone marrow samples

50 Umbilical cord samples

19 Olfactory mucosa samples

Optimization of Cell Isolation Procedures

Umbilical Cord

A variety of methods were utilized in order to liberate the endothelial cells from the lining of the umbilical cord vein. These methods included trypsin, accumax, tripLE and collagenase at various concentrations, singly and in different combinations for a variety of incubation periods (30 min to overnight). Also two different isolation methods were trialed. 1) Flushing method; whereby the cord was kept intact and filled with cell liberation enzymes followed by flushing of loosened cells from the cord. 2) Chopping method; whereby the cord was chopped into small pieces and incubated in cell liberating enzymes followed by collection of dissociated cells.

Of the two methods, the flushing method liberated the most cells. However, regardless of the method, very few cells were loosened from processed umbilical cords and many failed to attach and proliferate. In the most successful cultures, cells were able to proliferate until confluence. However at the first passage the majority of umbilical cord cultures failed to proliferate. Low glucose media has been reported in some literature to aid umbilical cord cell proliferation. Therefore this media was tested as a potential improvement to the cultures; however it failed to increase the proliferation of umbilical cord cells.

Bone Marrow

Bone marrow cultures were established from CD34 depleted bone marrow samples. In accordance with most literature, the cells were ficoll treated to remove red blood cells, washed and plated at high density. The vast majority of haematopoietic cells failed to attach within 48 hrs. The cultures were then washed, to remove unattached cells, and the remaining attached cells propagated. The bone marrow cultures were highly successful. Attached cultures were quickly established and proliferated rapidly.

Olfactory Mucosa

Initially olfactory mucosa cultures were established using explant techniques. This method involves chopping the tissue into small pieces, facilitating the attachment of the pieces to the substrate and subsequently allowing cells to migrate out from the explant and proliferate. Next, a dissociation method was utilized whereby the olfactory epithelium was separated from the underlying lamina propria using dispase. The lamina propria was dissociated with collagenase and the mucosa dissociated manually by trituration. More recently, a technique has been developed involving the incubation of chopped tissue pieces overnight in collagenase followed by trypsin digestion. To varying degrees, all methods used have been successful in the establishment of proliferative dissociated cultures.

Characterization of Dissociated Cultures

Immuno-fluorescence

To determine their phenotype, dissociated cultures derived from umbilical cord (2 samples), bone marrow (2 samples) and olfactory mucosa (8 samples) were immuno-stained for mesenchymal stem cell markers, as well as neural markers.

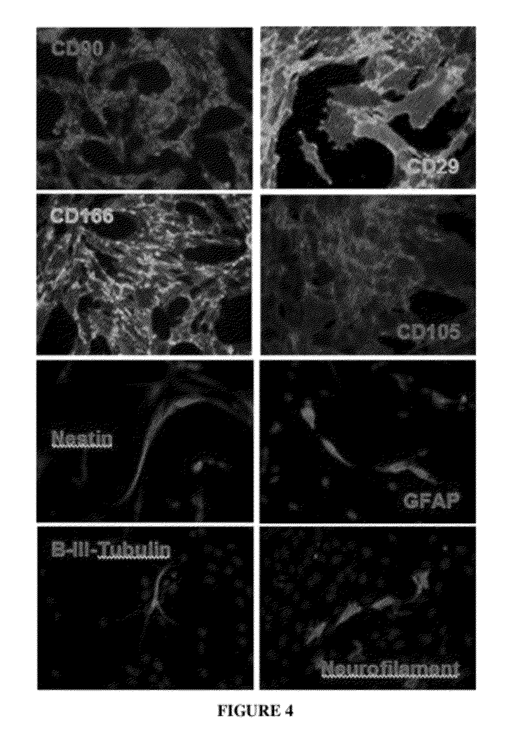

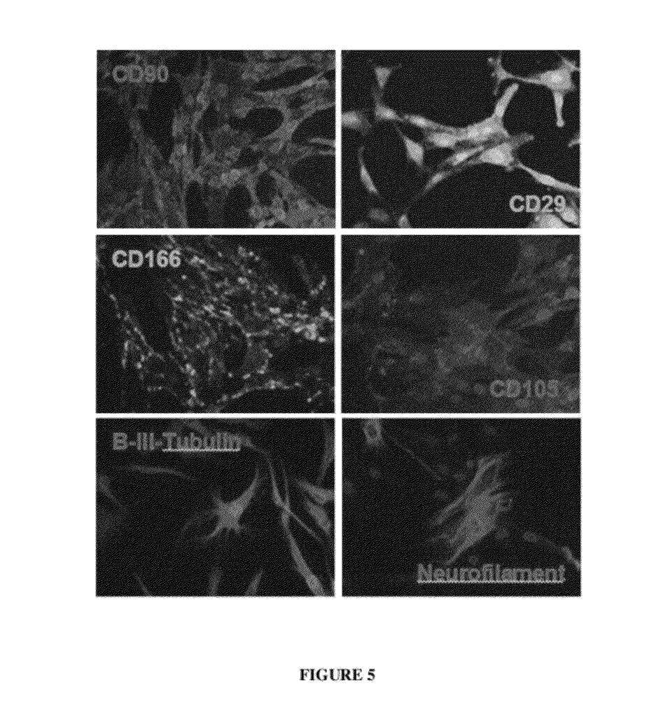

From these experiments it was observed that the olfactory mucosa cultures expressed both neural and mesenchymal stem cell markers (FIG. 4) and had a very similar phenotype to both the umbilical cord and bone marrow cultures (FIG. 5), known sources of mesenchymal stem cells (Table 4).

TABLE 4
Phenotype of olfactory mucosa (OLF) cultures compared
with bone marrow (BM) and umbilical cord (UC).

| Marker | OLF | BM | UC |
|---|---|---|---|
| CD29 | + | + | + |
| CD166 | + | + | + |
| CD105 | + | + | + |
| CD73 | + | + | + |
| CD90 | + | + | + |
| β-III-Tubulin | + | + | + |
| Neurofilament | + | + | + |
| Nestin | + | − | + |
| GFAP | + | − | + |

Surprisingly, it was also apparent that the bone marrow and umbilical cord cultures also contained cells that were positive for neural markers. Accordingly in certain embodiments, aspects of the invention relate to isolating mesenchymal and/or neural stem cells from olfactory mucosa, bone marrow, and/or umbilical cord.

Fluorescence Activated Cell Sorting (FACS)

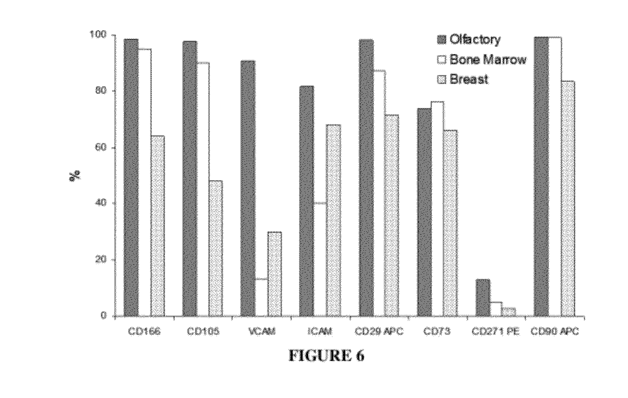

To further examine the phenotype of the dissociated cultures, and to provide percentages of positive cells, FACS analysis was performed on umbilical cord (1 sample), bone marrow (2 samples) and olfactory mucosa (8 samples) (FIG. 6). In addition, FACS analysis was also carried out on breast tissue samples (FIG. 6). Breast tissue was utilized because, like the olfactory mucosa, it is a solid tissue that contains epithelial cells. Therefore, the phenotypic analysis of breast tissue would help confirm results acquired for olfactory mucosa samples.

These experiments largely confirmed the previous immuno-fluorescence data. Except for the percentage of cells stained positive for VCAM and ICAM, the phenotype of bone marrow and olfactory mucosa cultures seem remarkably similar. Accordingly, in some embodiments, aspects of the invention relate to using either VCAM and/or ICAM alone or in combination with one or more additional markers described herein as discriminating markers between the two tissue types.

Expression of Mesenchymal Stem Cell Markers in Olfactory Mucosa Sections

Olfactory mucosa biopsies were snap-frozen, sectioned and stained for the expression of both neural markers and mesenchymal stem cell markers.

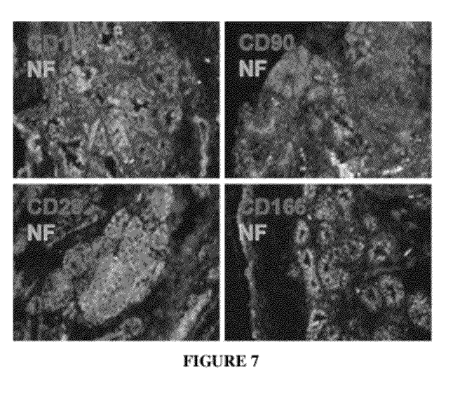

From these experiments it was shown that the olfactory mucosa expresses all the stem cell markers examined. However, there were variations in the amount of staining depending on the particular marker (FIG. 7). Some markers such as CD105 were only found in limited areas, whereas others, like CD29 or CD90 were widely expressed. Interestingly the expression of CD166 was determined to be largely, restricted to the areas surrounding nerve bundles and also co-localized with GFAP. This is significant in that it indicates that CD166 may be exclusively expressed in the olfactory mucosa by olfactory ensheathing cells. Of particular interest for this study, olfactory ensheathing cells have been used in both animal models and clinical trials of spinal cord injury. Therefore a cell extraction process, based on the expression of CD166, may be possible. Accordingly, in some embodiments, aspects of the invention relate to isolating olfactory ensheathing cells from olfactory mucosa (e.g., using FACS or other isolating method, for example, involving CD166 antibodies to identify and/or purify the ensheathing cells). Isolated ensheathing cells may be propagated. Isolated ensheathing cells (either propagated or non-propagated) may be used in a cell therapy (e.g., by injecting them into a subject at a site of spinal cord injury, or at a site of other injury or tissue disease).

Mesenchymal Stem Cell Differentiation

Mesenchymal stem cells, by definition, must be able to differentiate into, adipocytes, chondrocytes and osteocytes. The tissue cultures were evaluated to determine the extent to which they were able to function as mesenchymal stem cells and differentiate into these phenotypes.

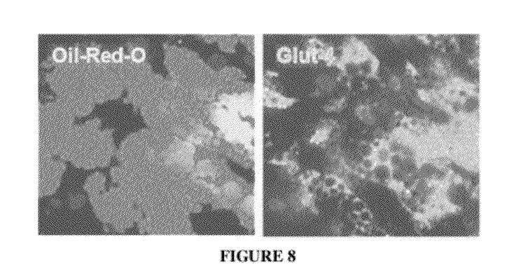

The differentiation assays were repeated 4 times on bone marrow and olfactory cultures. Bone marrow cultures were used as positive controls as they are a known source of mesenchymal stem cells. Bone marrow cells were able to differentiate into adipocytes indicated by the expression of the adipocyte marker Glut-4 and the accumulation of lipid indicated by oil-red-O staining (FIG. 8). However, no adipocytes were observed in olfactory cultures. Methods for differentiation of bone marrow cells into chondrocytes and osteocytes may be applied to olfactory cells, e.g., to gauge their response.

### REFERENCES

1: Pierelli, L et al. (2001) 42(6):1195

2: Arai, F et al. J Exp Med 195(12):1549

3: Dvorakova, J et al. (2008) Cell Biol Int 32(9):1116

4: Herrera, M B et al. (2006) Stem Cells 24(12):2840

5: Clark, B R et al. (1995) Ann N Y Acad Sci 29;770:70

6: Frederiksen, K. et al. (1988) Neuron 1:439.

7: Cattaneo, C. et al. (1990) Nature 347:762.

8: Reynolds, B. A. et al. (1992) Science 255:1707.

9: Rietze, R. L. et al. (2001) Nature 412:736.

10: Carpenter, M. K. et al. (2001) Exp. Neurol. 172:383

11: von Bohlen Und Halbach O, Cell Tissue Res 329(3):409

12: Eng, L F et al. (2000) Neurochem Res 25(9-10):1439

All publications, patents and sequence database entries mentioned herein, including those items listed below, are hereby incorporated by reference in their entirety as if each individual publication or patent was specifically and individually indicated to be incorporated by reference. In case of conflict, the present application, including any definitions herein, will control.

1. A method, comprising
obtaining an olfactory mucosa sample of a subject, and
obtaining a population of pluripotent stem cells from said sample.

2. The method of claim 1, wherein said olfactory mucosa sample is obtained from the middle turbinate, from the superior turbinate, or from the septum.

3-9. (canceled)

10. The method of claim 1, wherein the cells are enriched by cell sorting for or against any of the antigens CD105, CD166, CD90, CD29, Stro1, Tubulin, Neurofilament, GFAP, or Nestin or any combination of these.

11. The method of claim 1,
wherein said pluripotent stem cell population is able to self-renew over at least 10 cell passages, and/or
wherein cells of said pluripotent stem cell population are able to differentiate into cells of neuronal lineage or mesenchymal lineage or both.

12. (canceled)

13. The method of claim 1, wherein said subject is a human subject.

14. (canceled)

15. The method of claim 1, further comprising
differentiating cells of said pluripotent stem cell population into differentiated cell types; and/or
administering to a human subject indicated to have a disease or condition cells of said pluripotent stem cell population or cells differentiated from said pluripotent stem cell population, optionally, wherein the donor and the recipient of said cells is the same subject.

16-19. (canceled)

20. A pluripotent stem cell population obtained from the olfactory mucosa of a subject.

21. The stem cell population of claim 20, comprising cells expressing mesenchymal stem cell marker genes, and/or cells expressing neural stem cell marker genes.

21-22. (canceled)

23. The pluripotent stem cell population of claim 20, comprising cells expressing any of the following genes CD105, CD166, CD90, CD29m, Tubulin, Neurofilament, GFAP, Nestin, or any combination of them, optionally, wherein said cells are negative for Stro 1 expression in an immunoassay.

24-25. (canceled)

26. The pluripotent stem cell population of claim 20,
wherein cells of said pluripotent stem cell population are able to form neurospheres, are able to differentiate into cells of the mesenchymal, and/or neuronal lineage, optionally, wherein said differentiated cell types comprise adipocytes, osteocytes, chondrocytes, neuronal cells, or glial cells.

27-30. (canceled)

31. The pluripotent stem cell population of claim 20, wherein said stem cell population expresses one or more of the genes CD105, CD166, CD90, CD29, tubulin, neurofilament, GFAP, and nestin.

32. The pluripotent stem cell population of claim 20, wherein said stem cell population expresses
at least the genes CD105, CD166, and CD90,
at least the genes tubulin, neurofilament, and GFAP,
at least the genes tubulin, neurofilament, GFAP and nestin,
at least the genes GFAP and nestin, or
at least the genes tubulin, GFAP and nestin.

33-38. (canceled)

39. A pluripotent stem cell obtained from the olfactory mucosa of a subject.

40-41. (canceled)

42. The stem cell of claim 39, expressing any of the following genes CD105, CD 166, CD90, CD29m, Tubulin, Neurofilament, GFAP, Nestin, or any combination of them, and, optionally, negative for Stro 1 expression in an immunoassay, and/or able to differentiate into cells of the mesenchymal and/or neuronal lineage.

40-56. (canceled)

57. A composition comprising a pluripotent stem cell population obtained by the method of claim 1.

58-59. (canceled)

60. A method of treating a subject, comprising administering to said subject one or more of the pluripotent stem cell populations obtained by the method of claim 1.

61. (canceled)

62. The method of claim 60, wherein the subject is indicated to have a condition or disease, optionally, wherein the disease is
a degenerative disease,
a spinal cord injury,
a neurodegenerative disease,
a bone disease or dysfunction, or
a cartilage disease or dysfunction.

63-67. (canceled)

68. The method of claim 60, wherein the donor and the recipient of said cells of said pluripotent stem cell population are the same subject.

69-74. (canceled)

75. A method, comprising using one or more of the pluripotent stem cell populations obtained by the method of claim 1 in the therapy of a condition or disease.
